(19) United States
(12) Patent Application Publication
(10) Pub. No.: US 2022/0411765 A1
(43) Pub. Date: Dec. 29, 2022

(54) TRUNCATED HYALURONAN SYNTHASE AND POLYNUCLEOTIDE ENCODING THE SAME

(71) Applicants: Malihe Keramati, Tehran (IR); Reza Ahangari Cohan, Tehran (IR); Parsa Parsian, Tehran (IR); Elnaz Afshari, Tehran (IR)

(72) Inventors: Malihe Keramati, Tehran (IR); Reza Ahangari Cohan, Tehran (IR); Parsa Parsian, Tehran (IR); Elnaz Afshari, Tehran (IR)

(73) Assignees: Malihe Keramati, Tehran (IR); Reza Ahangari Cohan, Tehran (IR)

(21) Appl. No.: 17/844,351

(22) Filed: Jun. 20, 2022

Related U.S. Application Data
(60) Provisional application No. 63/212,669, filed on Jun. 20, 2021.

(51) Int. Cl. C12N 9/10

(52) U.S. Cl. CPC C12N 9/1051; C12Y 204/01212

(57) ABSTRACT

A truncated hyaluronan synthase (HAS) comprising an exemplary amino acid sequence having at least 95% identity with SEQ ID NO: 2. The truncated HAS may be encoded by an exemplary nucleic acid sequence set forth in SEQ ID NO: 3.

### CROSS-REFERENCE TO RELATED APPLICATION

This application claims the benefit of priority from U.S. Provisional Patent Application Ser. No. 63212669, filed on Jun. 20, 2021, entitled “TRUNCATED HYALURONAN SYNTHASE AND POLYNUCLEOTIDES ENCODING THE SAME” which is incorporated herein by reference in its entirety.

### TECHNICAL FIELD

The present disclosure is generally related to a truncated form of hyaluronan synthase (HAS), polynucleotides encoding the same, methods for producing the same, and compositions comprising the same, and more particularly to a truncated form of HAS that may lack all membrane associated domains (MDs) of an exemplary native full-length HAS.

### BACKGROUND

Hyaluronic acid (HA) is a glycosaminoglycan synthesized by the enzyme hyaluronan synthase (HAS) which may be localized inside the plasma membrane of prokaryotic and/or eukaryotic cells. It is believed that HA synthesis in these organisms may be a multi-step process including: i) an initiation step which may involve binding of an initial precursor, i.e., glucuronic acid (GlcA) and N-acetylglucosamine (GlcNAc), to the enzyme, ii) an elongation step that may involve alternate addition of GlcA and GlcNAc to the growing oligosaccharide chain, and iii) an extrusion step in which the growing polymer may be extruded across the plasma membrane and enter the extracellular space. According to the literature, almost all HAS proteins may share common predicted structural features, including a large central domain and clusters of two or more membrane-associated or transmembrane domains at both of the carboxyl and amino termini of the protein. The central domain may constitute up to ≈88% of intracellular HAS protein sequences and may include the catalytic regions of the enzyme.

HA has been found in almost all vertebrate tissues—most notably as an intra-articular matrix supplement—and has achieved widespread uses in different clinical applications. Several approaches have been proposed for producing HA, including—but not limited to—extraction from animal tissues, isolation from bacterial or eukaryotic cultures, and in vitro production. The in vitro method may produce HA using isolated HAS proteins (i.e., full-length proteins). A critical challenge with using isolated HAS may be its complicated isolation/purification process. The presence of two or more membrane associated domains in HAS structure may lead to many complexities in HAS expression, solubilization, and purification, and may limit the activity of enzyme to its localization inside the plasma membrane.

With this context, there is need to develop new structures of active HAS that may contribute to production of relatively large amounts of HA (with a certain molecular weight and narrow polydispersity), irrespective of being localized in the plasma membrane of an organism or fragments thereof.

### SUMMARY

This summary is intended to provide an overview of the subject matter of the present disclosure, and is not intended to identify essential elements or key elements of the subject matter, nor is it intended to be used to determine the scope of the claimed implementations. Its sole purpose is to present some concepts of one or more aspects in a simplified form as a prelude to the more detailed description that is presented later. The proper scope of the present disclosure may be ascertained from the claims set forth below in view of the detailed description below and the drawings.

One general aspect describes an exemplary truncated hyaluronan synthase (HAS). In one or more exemplary embodiments, exemplary truncated HAS may comprise a plurality of truncations, compared to an exemplary native full-length HAS that may have an exemplary amino acid sequence set forth in SEQ ID NO: 1. In one or more exemplary embodiments, the plurality of truncations may include an exemplary truncation of amino acid residues 1 to 56 of SEQ ID NO: 1 and an exemplary truncation of amino acid residues 216 to 417 of SEQ ID NO: 1. In an exemplary embodiment, an exemplary truncated HAS may comprise an exemplary amino acid sequence set forth in SEQ ID NO: 2. In an exemplary embodiment, exemplary truncated HAS may be encoded by an exemplary nucleic acid sequence set forth in SEQ ID NO: 3.

An exemplary embodiment may be directed to an exemplary truncated HAS which may have an exemplary amino acid sequence set forth in SEQ ID NO: 2.

This Summary may introduce a number of concepts in a simplified format; the concepts are further disclosed within the “Detailed Description” section. This Summary is not intended to configure essential/key features of the claimed subject matter, nor is intended to limit the scope of the claimed subject matter.

### BRIEF DESCRIPTION OF THE DRAWINGS

The novel features which are believed to be characteristic of the present disclosure, as to its structure, organization, use and method of operation, together with further objectives and advantages thereof, will be better understood from the following drawings in which an exemplary embodiment will now be illustrated by way of example. It is expressly understood, however, that the drawings are for the purpose of illustration and description only and are not intended as a definition of the limits of the present disclosure. Exemplary embodiments will now be described by way of example in association with the accompanying drawings in which:

FIG. 1 shows an exemplary schematic structure of an exemplary native full-length hyaluronan synthase (HAS) of Group G Streptococccus (GGS, in particular, Streptococcus dysgalactiae subspecies equisimilis), consistent with one or more exemplary embodiments of the present disclosure;

FIG. 2 shows an exemplary process flow diagram of an exemplary method for producing an exemplary truncated HAS set forth in SEQ ID NO: 2, consistent with one or more exemplary embodiments of the present disclosure;

FIG. 3 shows agarose gel electrophoresis of an exemplary polymerase chain reaction (PCR) conducted for amplifying an exemplary full-length HAS gene of GGS isolate S88 (GGS-S88) set forth in SEQ ID NO: 4, consistent with one or more exemplary embodiments of the present disclosure;

FIG. 4 illustrates agarose gel electrophoresis of an exemplary colony PCR conducted on exemplary Escherichia coli Top10 (E. coli Top10) colonies transformed with an exemplary recombinant pET21a(+) vector (harboring exemplary GGS-S88 HAS gene set forth in SEQ ID NO: 4), consistent with one or more exemplary embodiments of the present disclosure;

FIG. 5 shows restriction map analysis of exemplary recombinant plasmids (i.e., exemplary pET21a(+) harboring exemplary GGS-S88 HAS gene set forth in SEQ ID NO: 4) using NdeI and XhoI restriction enzymes, to verify insertion of exemplary GGS-S88 HAS gene into an exemplary pET21a(+) vector, consistent with one or more exemplary embodiments of the present disclosure;

FIG. 6 shows topology prediction result for exemplary native full-length HAS (i.e., exemplary GGS-S88 HAS set forth in SEQ ID NO: 1) obtained using the TMHMM server, consistent with one or more exemplary embodiments of the present disclosure;

FIG. 7 illustrates agarose gel electrophoresis of an exemplary PCR conducted for amplification of an exemplary truncated HAS gene (SEQ ID NO: 3), consistent with one or more exemplary embodiments of the present disclosure;

FIG. 8 illustrates agarose gel electrophoresis of an exemplary colony PCR conducted on exemplary E. coli BL21 (DE3) colonies transformed with exemplary pET21a(+) vectors harboring exemplary truncated HAS gene (SEQ ID NO: 3), consistent with one or more exemplary embodiments of the present disclosure;

FIG. 9 shows restriction map analysis of exemplary recombinant plasmids (i.e., exemplary pET21a(+) vectors harboring exemplary truncated HAS gene), using NdeI and XhoI restriction enzymes, to verify insertion of exemplary truncated HAS gene (SEQ ID NO: 3) into exemplary pET21a(+) vector, consistent with one or more exemplary embodiments of the present disclosure;

FIG. 10A shows SDS-PAGE (Sodium dodecyl-sulfate polyacrylamide gel electrophoresis) analysis of exemplary purified full-length HAS and exemplary purified truncated HAS (SEQ ID Nos: 2 and 1, respectively) before IPTG induction, consistent with one or more exemplary embodiments of the present disclosure;

FIG. 10B shows SDS-PAGE analysis of exemplary purified full-length HAS and exemplary purified truncated HAS (SEQ ID NOs: 2 and 1, respectively) after IPTG induction, consistent with one or more exemplary embodiments of the present disclosure;

FIG. 11 shows western blot analysis of exemplary full-length HAS and exemplary truncated HAS (SEQ ID NOs: 2 and 1, respectively) after expression in an exemplary transformed E. coli BL21 (DE3), consistent with one or more embodiments of the present disclosure;

FIG. 12 shows western blot analysis of exemplary full-length HAS and exemplary truncated HAS (SEQ ID NOs: 2 and 1, respectively) after being eluted from an exemplary Ni²⁺-NTA column, consistent with one or more embodiments of the present disclosure; and

FIG. 13 shows an exemplary standard curve obtained based on measuring the absorbance (at 550 nm) of exemplary glucuronic acid standard solutions in an exemplary carbazole assay, consistent with one or more exemplary embodiments of the present disclosure.

### DETAILED DESCRIPTION

In the following detailed description, numerous specific details are set forth by way of examples to provide a thorough understanding of the relevant teachings related to the exemplary embodiments. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in one or more exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be plain to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Provided herein is an exemplary truncated hyaluronan synthase (HAS), an exemplary polynucleotide or exemplary polynucleotides encoding an exemplary truncated HAS, an exemplary method for producing an exemplary truncated HAS, an exemplary method for producing an exemplary polynucleotide encoding an exemplary truncated HAS, exemplary compositions comprising an exemplary truncated HAS, and exemplary compositions comprising an exemplary polynucleotide that may encode an exemplary truncated HAS. Furthermore, one or more exemplary embodiments may be directed to an exemplary vector that may harbor an exemplary polynucleotide encoding an exemplary truncated HAS. In one or more exemplary embodiments, exemplary host cells may carry an exemplary vector that may harbor an exemplary polynucleotide encoding an exemplary truncated HAS.

Hyaluronan synthase—also known as hyaluronate synthase, hyaluronic acid synthase, HA synthase, and HAS—may refer to a membrane-bound enzyme that may polymerize a glycosaminoglycan polysaccharide chain comprising alternating GlcNac and GleVa sugars. HAS may be involved in the hyaluronan biosynthesis pathway that is part of the Glycan biosynthesis pathway. HAS may be divided into two classes: i) Class I (comprising single-domain integral membrane protein), and ii) Class II (comprising two-domain membrane anchored/soluble protein). Each domain of Class II HAS may be responsible for catalysis of a type of glycosidic linkage. Though Class II HAS may be found solely in Pasturella multocida, Class I HAS may be ubiquitous and may have a more complex structure. Topology of Class I HAS (e.g., Streptococcal HAS) may comprise six membrane regions from which four may be integral, one may be a cytoplasmic domain, and two may be amphipathic. An exemplary native HAS may be found in both prokaryotes and eukaryotes. Common prokaryotic sources may include—but are not limited to—Group A, Group C, Group D, and/or Group G of Streptococcus sources (such as Streptococcus equisimilis, Streptococcus pyogenes, Streptococcus zooepidemicus, Streptococcus haemolyticus, and Streptococcus uberis) Sulfolobus solfataricus, and Pasteurella multocida. Some eukaryotes and viruses may also produce HA, including—but not limited to—Xenopus laevis and Paramecium bursaria Chlorella virus. Thereby, these sources may include HAS genes that may be employed in one or more exemplary embodiments. In one or more exemplary embodiments, eukaryotic sources may also include—but not limited to—dermal fibroblasts, synovial chondrocytes and fibroblasts, and trabecular-meshwork cells. In one or more exemplary embodiments, exemplary HAS gene of Group A, Group C, and/or Group G of Streptococcus may be used for producing an exemplary truncated HAS described in one or more exemplary embodiments.

In an exemplary embodiment, Group G of Streptococcus—including, but not limited to, Streptococcus dysgalactiae subspecies equisimilis, Streptococcus milleri, Streptococcus canis, and Streptococcus intestinalis—may be used for producing an exemplary truncated HAS disclosed in one or more exemplary embodiments. Group G streptococcus/streptococci or Group G beta-hemolytic streptococcal strains—abbreviated as GGS—may belong to heterogeneous species of streptococci that may comprise nonpathogenic commensals and/or pathogenic bacteria. The strains under Group G may generate severe infections, including bacteremia, and may commonly express a surface protein homologous to an exemplary M protein of Streptococcus pyogenes. GGS strains may include, but are not limited to, Streptococcus dysgalactiae subspecies equisimilis, Streptococcus milleri, Streptococcus canis, and Streptococcus intestinalis. Streptococcus may, taxonomically, be subdivided into Lancefield Groups according to different carbohydrate antigens of cell wall. In one or more exemplary embodiments, Streptococcus may be subdivided into 20 distinct Lancefield groups, however the most clinically relevant groups may include A, B, C, G, D, and Viridans (which may not conform to any specific Lancefield groups). The cell-wall carbohydrates of an exemplary GGS may be found in several β-hemolytic streptococcal species—including, but not limited to, Streptococcus canis and Streptococcus anginosus. In an exemplary embodiment, cell-wall carbohydrates of an exemplary GGS may be found in Streptococcus dysgalactiae sub species equisimilis.

In one or more exemplary embodiments, an exemplary truncated HAS may be capable of synthesizing different amounts of HA (i.e., larger or smaller amounts of HA) and different sizes of HA (i.e., larger or smaller HA in size/molecular weight), compared to the produced HA by an exemplary native full-length HAS. In one or more exemplary embodiments, an exemplary truncated HAS may produce an exemplary HA with amounts and sizes similar to the HA produced by an exemplary native full-length HAS. “Hyaluronic acid (HA)” may be a linear polysaccharide of repeating disaccharide units—with a high molecular weight—consisting essentially of glucuronic acid (GlcA) and N-acetylglucosamine (GlcNAc). The number of repeating disaccharide units in an exemplary HA molecule may exceed 30,000. HA may be the only glycosaminogylcan that may be synthesized by both mammalian cells and prokaryotes. HA may be also known as Hyaluronan and/or hyaluronate.

An exemplary truncated HAS, disclosed in one or more exemplary embodiments, may include a plurality of exemplary truncations compared to a corresponding native full-length HAS. “Native” may refer to an exemplary allele, an exemplary gene (an exemplary full-length gene), or an exemplary protein/polypeptide that may be the most frequently observed phenotype and/or genotype in nature. Hence, a native gene may be an exemplary sequence (either nucleic acid or amino acid/protein sequence) normally found in an exemplary organism in its native or wild state. “Truncation” may refer to elimination of one or more segments/fragments of an exemplary polypeptide (e.g., an exemplary protein) and/or an exemplary polynucleotide (e.g., an exemplary gene). Thus, an exemplary truncated gene, protein, and/or enzyme may lack one or more exemplary truncations (i.e., exemplary amino acid and/or nucleic acid sequence of a gene, protein, or enzyme may lack one or more segments/fragments).

In one or more exemplary embodiments, an exemplary truncated HAS may lack one or more membrane-associated domains (MDs) compared to a corresponding native full-length HAS. “Membrane-associated domain (MDs)” may refer to a fragment/segment of a protein that may be attached to, or associated with, or coupled to, a cell membrane (i.e., a fragment/segment may be located/disposed inside the plasma membrane of a cell or may surround a cell). In particular, membrane-associated domain may include any of extracellular, intracellular, and/or integral fragment/segment of a protein that may be attached to, or associated with, or coupled to, a cell membrane.

In one or more exemplary embodiments, an exemplary truncated HAS may further lack one or more segments of an exemplary intracellular region and/or an exemplary extracellular region of a corresponding native full-length HAS. In an exemplary embodiment, an exemplary truncated HAS may have no MDs—compared to a corresponding native full-length HAS—and may lack a plurality of segments of exemplary intracellular region and/or exemplary extracellular region of a corresponding native full-length HAS. Thereby—due to having no MDs—an exemplary truncated HAS may have enzyme activity without needing to be disposed/localized inside plasma membrane. In one or more exemplary embodiments, an exemplary truncated HAS may have an altered enzyme activity in comparison to a corresponding native full-length HAS. In an exemplary embodiment, an exemplary truncated HAS may be used for producing an exemplary HA by an exemplary in vitro method. An exemplary in vitro method—compared to other methods including but not limited to HA extraction from animal tissues and HA isolation from bacterial or eukaryotic cultures—may produce an exemplary HA (with low polydispersity and a defined molecular weight) using an exemplary isolated HAS protein.

In one or more exemplary embodiments, an exemplary truncated HAS may lack all MDs and a plurality of segments of exemplary intracellular and extracellular regions that are structurally found in a corresponding native full-length HAS. In one or more exemplary embodiments, native full-length HAS, native HAS, and/or full-length HAS may refer to an exemplary Class I native full-length HAS. In particular, consistent with one or more exemplary embodiments, an exemplary native full-length HAS may refer to an exemplary native full-length HAS of a Streptococci species including—but not limited to—Lancefield Groups A, B, C, G, and/or D. In an exemplary embodiment, an exemplary native full-length HAS may refer to an exemplary native HAS of Group G Streptococcus (GGS). The GGS may include—but is not limited to—Streptococcus canis, Streptococcus anginosus, and Streptococcus dysgalactiae subspecies equisimilis. In an exemplary embodiment, an exemplary native full-length HAS may refer to an exemplary native HAS of Streptococcus dysgalactiae subspecies equisimilis (Group G) that may have an exemplary amino acid sequence set forth in SEQ ID NO: 1 (also set forth in GenBank: QSG30229.1). Furthermore, an exemplary native full-length HAS may refer to one or more exemplary amino acid sequence with at least 80%, at least 85%, at least 90%, at least 95%, and/or at least 98.5% sequence identity to SEQ ID NO: 1.

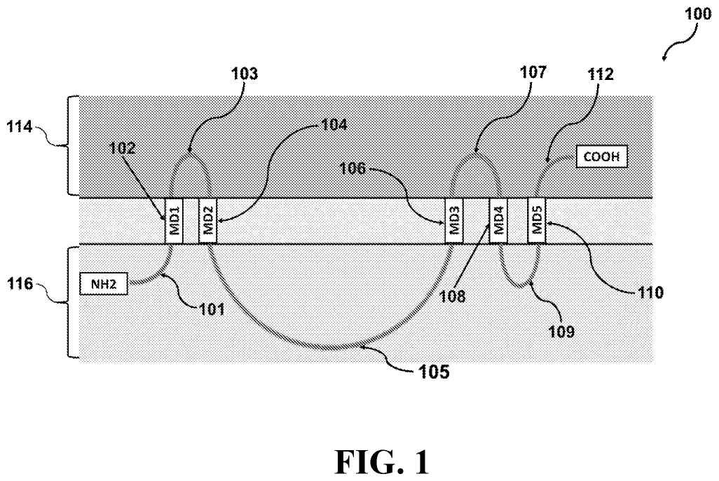

FIG. 1 shows an exemplary schematic structure 100 of an exemplary native full-length HAS of GGS (in particular Streptococcus dysgalactiae subspecies equisimilis), consistent with one or more exemplary embodiments of the present disclosure. As shown in FIG. 1, exemplary native full-length HAS of GGS may include five MDs, three extracellular regions (localized outside 114 cell), and three intracellular regions (localized inside 116 cell). In one or more exemplary embodiments, i) residues 1-6, 57-319, and 372-377 of SEQ ID NO: 1 may, respectively, compose an exemplary first, an exemplary second, and an exemplary third intracellular region of exemplary native full-length HAS (SEQ ID NO: 1); ii) residues 7-29, 34-56, 320-342, 352-371, and 378-395 may, respectively, compose an exemplary first, an exemplary second, an exemplary third, an exemplary fourth, and an exemplary fifth membrane-associated domain (MD) of exemplary native full-length HAS (SEQ ID NO: 1); and iii) residues 30-33, 343-351, and 396-417 may, respectively, compose an exemplary first, an exemplary second, and an exemplary third extracellular region of exemplary native full-length HAS (SEQ ID NO: 1). In one or more exemplary embodiments, exemplary first membrane-associated domain, exemplary second membrane-associated domain, exemplary third membrane-associated domain, exemplary fourth membrane-associated domain, and exemplary fifth membrane-associated domain may be referred to as MD1, MD2, MD3, MD4, and MDS, respectively. Referring again to FIG. 1, MD1, MD2, MD3, MD4, and MD5 are labeled as 102, 104, 106, 108, and 110, respectively. Exemplary first intracellular region, exemplary second intracellular region, and exemplary third intracellular region are labeled as 101, 105, and 109, respectively. Exemplary first extracellular region, exemplary second extracellular region, and exemplary third extracellular region are labeled as 103, 107, and 112, respectively. As shown in FIG. 1, MDs are numbered as 1 to 5, starting from an exemplary N-terminus of exemplary native full-length HAS (SEQ ID NO: 1). “Terminus” or “termini” may refer to an exemplary end of an exemplary polynucleotide or polypeptide. An exemplary end of an exemplary polynucleotide or polypeptide may not be limited only to exemplary final or first sites of a polynucleotide or polypeptide, but may include extra nucleotides or amino acids in exemplary terminal regions of a polynucleotide or polypeptide. A polypeptide may have both a C-terminus (terminated by an amino acid with a free carboxyl group (COOH)) and an N-terminus (terminated by an amino acid with a free amino group (NH₂)). In an exemplary embodiment, exemplary C-termini and/or N-termini of exemplary polypeptides may be modified such that they terminate or begin with an exemplary non-polypeptide moiety like an exemplary organic conjugate.

In one or more exemplary embodiments, exemplary truncated HAS may have an amino acid sequence of about 159 amino acids. In an exemplary embodiment, exemplary truncated HAS may comprise amino acids 57 to 215 of exemplary native full-length HAS (SEQ ID NO: 1) and, in particular, may include exemplary amino acid sequence set forth in SEQ ID NO: 2. In one or more exemplary embodiments, exemplary truncated HAS (SEQ ID NO: 2), compared to exemplary native full-length HAS (SEQ ID NO: 1), may include an exemplary truncation of amino acid residues 1 to 56 and an exemplary truncation of amino acid residues 216 to 417 of SEQ ID NO: 1.

Thereby, in one or more exemplary embodiments, exemplary truncated HAS (SEQ ID NO: 2) may lack amino acid residues 1 to 6 (composing exemplary first intracellular region 101), 7 to 29 (composing exemplary MD1 (102)), 30 to 33 (composing exemplary first extracellular region 103), 34 to 56 (composing exemplary MD2 (104)), 216 to 319 (composing an exemplary fragment of exemplary second intracellular region 105), 320 to 342 (composing exemplary MD3 (106)), 343 to 351 (composing exemplary second extracellular region 107), 352 to 371 (composing exemplary MD4 (108)), 372 to 377 (composing exemplary third intracellular region 109), 378 to 395 (composing exemplary MD5 (110)), and 396 to 417 (composing exemplary third extracellular region 112) of exemplary native full-len gth HAS set forth in SEQ ID NO: 1 (i.e., exemplary native full-length HAS of GGS shown in FIG. 1). Thus, in one or more exemplary embodiments, exemplary truncated HAS (SEQ ID NO: 2) may lack amino acid residues 1 to 56 and amino acid residues 216 to 417 of SEQ ID NO: 1. In an exemplary embodiment, exemplary truncated HAS (SEQ ID NO: 2) may include amino acid residues 57 to 215 of exemplary native full-length HAS (SEQ ID NO: 1).

In one or more exemplary embodiments, an exemplary truncated HAS may include an exemplary amino acid sequence with at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% sequence identity to SEQ ID NO: 2. In an exemplary embodiment, an exemplary amino acid sequence may be at least 95 to 99.5% identical to SEQ ID NO: 2. In one or more exemplary embodiments, exemplary truncated HAS set forth in SEQ ID NO: 2 may have a molecular weight of about 30 kDa (kiloDalton) compared to exemplary native full-length HAS of GGS (SEQ ID NO: 1) that may have a molecular weight of about 42 kDa. “Identity” and “identical” may refer to a relationship between exemplary nucleic acid sequences of two or more polynucleotides that may be determined by comparing exemplary sequences. As appreciated by those skilled in the art, identity may also refer to an exemplary degree of relationship between the sequences calculated by determining number of matches between residues of two or more nucleic acid strings. Identity may be obtained by calculating the number of identical matches between two or more exemplary sequences (preferably the smaller one) by further considering gap alignments (if any) addressed by a computer program (e.g., algorithms) or a particular mathematical model. % Identity applied to two or more exemplary polynucleotide sequences may refer to the percentage of residues (i.e., nucleic acid residues) in an exemplary candidate nucleic acid sequence that may be identical to the residues in an exemplary second nucleic acid sequence after aligning exemplary sequences and gap alignment, if necessary, to achieve a maximum percent identity. In one or more exemplary embodiments, exemplary variants of an exemplary polynucleotide (i.e., an exemplary reference polynucleotide) may have at least 40% to at least 99%, but less than 100%, sequence identity to an exemplary reference polynucleotide.

In one or more exemplary embodiments, an exemplary truncated HAS may also include an exemplary amino acid sequence with at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% sequence homology to SEQ ID NO: 2. In an exemplary embodiment, an exemplary amino acid sequence may be at least 95 to 99.5% homologous to SEQ ID NO: 2. “Homology” may mean that exemplary aligned/compared sequences may have diverged in evolution from a common origin. Homolog may imply that, for example, a first nucleic acid sequence or amino acid sequence (e.g., protein or gene (DNA or RNA) sequence) may be related to a second nucleic acid sequence or amino acid sequence by originating from a common ancestral sequence. Homolog may also refer to a relationship between proteins and/or genes that may be separated by speciation, or to a relationship between proteins and/or genes separated by genetic duplication. Two exemplary nucleic acid sequences may be considered homologous if exemplary polypeptides they encode include one or more stretch of at least 20 amino acids that may be at least 50%, 60%, 70%, 80%, 90%, 95%, or even 99% identical.

In one or more exemplary embodiments, an exemplary polynucleotide/nucleic acid molecule encoding an exemplary truncated HAS set forth in SEQ ID NO: 2 is disclosed. In one or more exemplary embodiments, exemplary polynucleotide may include an exemplary open reading frame (ORF) that may encode an exemplary truncated HAS (SEQ ID NO: 2). “Open reading frame” or “ORF” may refer to a series of nucleotide triplets, where each triplet may encode a specific amino acid residue. In brief, an exemplary ORF may make a sequence translatable into an exemplary polypeptide or protein. An exemplary polynucleotide may include—but is not limited to—RNA and single-stranded or double-stranded DNA. In one or more exemplary embodiments, an exemplary polynucleotide and/or an exemplary ORF may include an exemplary nucleic acid sequence set forth in SEQ ID NO: 3.

In an exemplary embodiment, an exemplary nucleic acid sequence encoding an exemplary truncated HAS (i.e., exemplary nucleic acid sequence of SEQ ID NO: 3) may lack exemplary nucleic acid segments encoding amino acid residues 1 to 6 (composing exemplary first intracellular region 101), exemplary MD1 (102), amino acid residues 30 to 33 (composing exemplary first extracellular region 103), exemplary MD2 (104), amino acid residues 216 to 319 (composing an exemplary fragment of exemplary second intracellular region 105), exemplary MD3 (106), amino acid residues 343 to 351 (composing exemplary second extracellular region 107), exemplary MD4 (108), amino acid residues 372 to 377 (composing exemplary third intracellular region 109), exemplary MD5 (110), and amino acid residues 396 to 417 (composing exemplary third extracellular region 112) of exemplary native full-length HAS (i.e., exemplary native full-length HAS of GGS shown in FIG. 1).

In one or more exemplary embodiments, an exemplary nucleic acid sequence encoding an exemplary truncated HAS (SEQ ID NO: 2) may encode exemplary amino acid residues 57 to 215 of SEQ ID NO: 1. In an exemplary embodiment, exemplary native full-length HAS of GGS (in particular exemplary native full-length HAS of Streptococcus dysgalactiae subspecies equisimilis) may have an exemplary nucleic acid sequence as set forth in SEQ ID NO: 4.

In one or more exemplary embodiments, exemplary polynucleotide and/or exemplary ORF encoding exemplary truncated HAS may include an exemplary nucleic acid sequence with at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% sequence identity to SEQ ID NO: 3. In an exemplary embodiment, an exemplary nucleic acid sequence may be at least 95 to 99.5% identical to SEQ ID NO: 3 genes. It is to be understood that a wide variety of exemplary nucleic acid molecules that may encode exemplary truncated HAS set forth in SEQ ID NO: 2 may be contemplated to be within the scope of one or more exemplary embodiments, even if their nucleic acid sequences are not explicitly addressed herein for the sake of conciseness. As such, any exemplary polynucleotide comprising exemplary insertions, additions, substitutions, covalent modifications, and/or deletions with respect to exemplary nucleic acid sequences, described in one or more exemplary embodiments, may be included within the scope of one or more exemplary embodiments.

In one or more exemplary embodiments, exemplary polynucleotides may comprise one or more exemplary modifications or segments (e.g., a sequence) that may lead to a desirable feature—such as increased stability, durability, expression, translation, etc.—and/or an additional moiety for subcellular targeting or tracking (e.g., a detectable label/tag like a fluorescent label), and a binding site for a protein or protein complex, etc. In one or more exemplary embodiments, exemplary modifications may include, but are not limited to, modifications that may provide other chemical groups having or incorporating additional charge, hydrophobicity, polarizability, electrostatic interaction, hydrogen bonding, and fluxionality to the polynucleotide bases or to the polynucleotide as a whole. In one or more exemplary embodiments, exemplary modifications may provide nuclease-resistant oligonucleotides; such exemplary modifications may include one or more substituted inter-nucleotide linkages, altered bases, altered sugars, and a combination thereof. Exemplary modifications may further include, but are not limited to, 5′-position pyrimidine modifications, 2′-position sugar modifications, modifications at exocyclic amines, 8-position purine modifications, substitution of 5-bromo or 5-iodo-uracil, and 4-thiouridine substitution, phosphorothioate or alkyl phosphate modifications, methylations, backbone modifications, and unusual base-pairing combinations such as isoguanidine and isobases isocytidine. Modifications may further comprise 5′ and 3′ modifications such as capping. Other non-limiting examples of modification may comprise addition of a 5′ cap (e.g., a 3′ polyadenylated tail (i.e., a 3′ poly-Adenosine tail), a 7-methylguanylate cap (m7G)), a stability control sequence, a riboswitch sequence (to allow for regulated accessibility by exemplary proteins and/or protein complexes and/or regulated stability), an exemplary sequence that may form a dsRNA secondary structure (e.g., a hairpin), an exemplary modification or sequence that may target an exemplary RNA to a subcellular location (such as mitochondria, nucleus, chloroplasts, etc.), an exemplary sequence or an exemplary modification that may generate an exemplary binding site for proteins (e.g., exemplary proteins that may perform an exemplary function when landing on a DNA—including transcriptional repressors, transcriptional activators, DNA methyltransferases, histone acetyltransferases, DNA demethylases, histone deacetylases, etc.), an exemplary sequence or modification that may be used for tracking (e.g., an exemplary sequence that may provide fluorescent detection, conjugation to a moiety that may provide fluorescent detection, direct conjugation to a fluorescent molecule, etc.), and combinations thereof

Exemplary polynucleotides may further comprise exemplary non-natural nucleotide(s). Exemplary non-natural nucleotide may refer to an artificially-constructed nucleotide that may resemble—in chemical properties and/or structure—to natural nucleotide. Examples of the non-natural nucleotide may include—but are not limited to—abasic nucleoside, arabinonucleoside, 2′-deoxyuridine, α-deoxyribonucleoside, β-L-deoxyribonucleoside, and other glycosylated nucleosides. Exemplary glycosylated nucleosides may include glycosylated nucleosides having substituted pentose (2′-O-methylribose, 2′-deoxy-2′-fluororibose, 3′-O-methyl ribose, or 1′,2′-deoxyribose), arabinose, substituted arabinose sugar, substituted hexose, or alpha anomer. Exemplary non-natural nucleoside may further include an artificially constructed base analog or an artificially constructed chemically-modified base. Examples of base analog may include a 2-oxo(1H)-pyridin-3-yl group, a 5-substituted 2-oxo(1H)-pyridin-3-yl group, a 2-amino-6-(2-thiazolyl)purin-9-yl group, and a 2-amino-6-(2-oxazolyl)purin-9-yl group. Examples of chemically-modified base may include modified pyrimidine (e.g., 5-hydroxycytosine, 5-fluorouracil and 4-thiouracil), modified purine (e.g., 6-methyladenine and 6-thioguanosine), and other heterocyclic bases.

It is appreciated that any exemplary nucleic acid sequences disclosed in one or more exemplary embodiments may be prepared synthetically, e.g., using a commercially available poly/oligo-nucleotide synthesizer. Methods of synthetic oligonucleotide synthesis include, but are not limited to, solid-phase oligonucleotide synthesis, liquid-phase oligonucleotide synthesis, and other techniques known in the art.

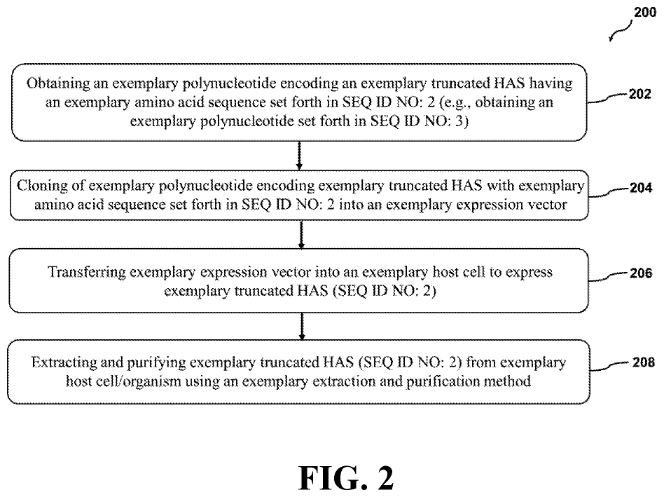

FIG. 2 shows an exemplary process flow diagram of exemplary method 200 for producing exemplary truncated HAS set forth in SEQ ID NO: 2, consistent with one or more exemplary embodiments of the present disclosure. Exemplary method 200 may include one or more steps with regards to exemplary aspects and embodiments described above. In an exemplary embodiment, exemplary method 200 may include: obtaining an exemplary polynucleotide encoding an exemplary truncated HAS having an exemplary amino acid sequence set forth in SEQ ID NO: 2 (e.g., obtaining an exemplary polynucleotide set forth in SEQ ID NO: 3) (step 202); cloning of exemplary polynucleotide encoding exemplary truncated HAS with exemplary amino acid sequence set forth in SEQ ID NO: 2 into an exemplary expression vector (step 204); transferring exemplary expression vector into an exemplary host cell to express exemplary truncated HAS (SEQ ID NO: 2) (step 206); and extracting and purifying exemplary truncated HAS (SEQ ID NO: 2) from exemplary host cell/organism using an exemplary extraction and purification method (step 208).

Referring to FIG. 2, step 202 may include obtaining exemplary polynucleotide encoding exemplary truncated HAS having exemplary amino acid sequence set forth in SEQ ID NO: 2 (e.g., obtaining exemplary polynucleotide set forth in SEQ ID NO: 3). In an exemplary embodiment, exemplary polynucleotide may have the nucleic acid sequence set forth in SEQ ID NO: 3. In an exemplary embodiment, exemplary polynucleotide may be amplified using exemplary nucleic acid sequence of SEQ ID NO: 4 as template and at least one pair of specific primers. For example, in order to obtain/amplify exemplary nucleic acid sequence of SEQ ID NO: 3, an exemplary forward primer set forth in SEQ ID NO: 7 and an exemplary reverse primer set forth in SEQ ID NO: 8 may be used. In one or more exemplary embodiments, exemplary polynucleotide encoding exemplary truncated HAS may be prepared synthetically, e.g., by using an exemplary commercially available oligo/gene synthesizers.

Step 204 may include cloning of exemplary polynucleotide encoding exemplary truncated HAS with exemplary amino acid sequence set forth in SEQ ID NO: 2 into exemplary expression vector. In an exemplary embodiment, step 204 may include cloning of exemplary polynucleotide set forth in SEQ ID NO: 3 into exemplary expression vector. In an exemplary embodiment, an exemplary molecular cloning technique may be employed to construct an exemplary recombinant vector harboring an exemplary nucleic acid (e.g., DNA) fragment of interest. Exemplary molecular cloning technique may include amplification of exemplary DNA fragment of interest before being inserted into an exemplary vector. An exemplary recombinant vector may then be transferred into an exemplary host cell/organism. In one or more exemplary embodiments, exemplary host cells may then be screened to find exemplary cells carrying the recombinant vector (that may harbor the DNA fragment of interest). “Vector” may refer to a tool that may facilitate transfer of a nucleic acid entity from one environment to another. An exemplary vector may be a replicon including—but not limited to—a phage, plasmid, or cosmid into which another DNA segment may have been inserted so as to provide replication of an exemplary inserted fragment. In one or more exemplary embodiments, an exemplary vector may be replicated when associated with one or more exemplary control elements. A vector may refer to an exemplary nucleic acid molecule that may be capable of transporting another nucleic acid molecule—to which it has been coupled—into a host cell. Exemplary vectors may include, but are not limited to: i) single-stranded, double-stranded, or partially double-stranded nucleic acid molecules, ii) nucleic acid molecules having no free ends (e.g., circular), or one or more free ends, iii) nucleic acid molecules comprising RNA, DNA, or both, and iv) other varieties of nucleic acid molecules. One type of vectors may include plasmid that may refer to an exemplary circular double-stranded DNA loop into which additional DNA fragments may be inserted/cloned, e.g., by exemplary standard molecular cloning techniques. Another type of vectors may include an exemplary viral vector comprising exemplary virally-derived RNA or DNA sequences which may be requisite for packaging into an exemplary virus (e.g., adenoviruses, replication defective retroviruses, retroviruses, adeno-associated viruses, and replication defective adenoviruses). Exemplary viral vectors may also include exemplary polynucleotides that may be commonly carried by viruses for transfection into an exemplary host cell. Some vectors may be capable of replicating autonomously in an exemplary host cell into which they have been transferred (e.g., episomal mammalian vectors and/or bacterial vectors/plasmids containing a bacterial origin of replication (Ori)). Other vectors, such as non-episomal mammalian vectors, may be integrated into genome upon entering into a host cell and may be replicated alongside an exemplary host cell's genome. Meanwhile, some vectors may direct expression of an exemplary inserted gene of interest; such vectors may be referred to as expression vectors in one or more exemplary embodiments. Plasmids may constitute one of the most commonly used expression vectors in recombinant DNA techniques. For example, an exemplary recombinant expression vector may harbor an exemplary polynucleotide, disclosed in one or more exemplary embodiments, in a form that may provide expression of exemplary nucleic acid molecule in an exemplary host cell. In one or more exemplary embodiments, an exemplary recombinant expression vector may include at least one exemplary regulatory element which may be selected based on an exemplary host cell used for expression. An exemplary recombinant expression vector may be operatively-linked to exemplary polynucleotide that is intended to be expressed. Within a recombinant expression vector, “operably linked” may refer to a state in which an exemplary polynucleotide of interest may be coupled to exemplary regulatory element(s) in a manner allowing for expression of an exemplary polynucleotide (e.g., in an exemplary host cell—when an exemplary vector is introduced into an exemplary host cell—or in an exemplary in vitro translation/transcription system). In one or more exemplary embodiments, an exemplary expression vector may further include one or more exemplary regulatory elements for controlling expression of an exemplary protein/polypeptide. Exemplary regulatory elements may include, but are not limited to, enhancers and promoters. “Enhancer” may refer to an exemplary DNA/nucleic acid sequence that may induce promoter activity and may be an innate element of a heterologous element or a promoter inserted to enhance the specificity of a promoter or transcription level.

In one or more exemplary embodiments, selection of an appropriate exemplary vector may depend on the type of exemplary host organism/cell into which an exemplary vector may be transferred. “Host organism/cell” may refer to an exemplary cell which may have been transformed or may be capable of being transformed, by an exemplary exogenous nucleic acid sequence. An exemplary host organism/cell may include, but is not limited to, eukaryotic cells such as yeast cells and insect cells, prokaryotic cells such as Escherichia coli (E. coli) cells, plant cells, and animal cells (e.g., mammalian cells, such as human cells, mouse cells, etc.). Being transformed may refer to transfer of an exemplary polynucleotide fragment into an exemplary host cell/organism, either in form of an exemplary plasmid that may be autonomously-replicating or may be integrated stably into the chromosome of an exemplary host cell/organism, resulting in genetically stable inheritance.

In an exemplary embodiment, an exemplary vector may be an autonomously replicating vector (i.e., a vector that may exist as an extra-chromosomal entity and may be capable of being replicated independent of chromosomal replication, e.g., an exemplary plasmid). In one or more exemplary embodiments, as stated above, an exemplary vector may be capable of being integrated into the genome of an exemplary host cell/organism—in part or in its entirety—and may be replicated along with an exemplary chromosome into which it has been integrated.

In one or more exemplary embodiments, an exemplary expression vector may further include a drug-resistance marker (e.g., an antibiotic-resistance marker), one or more origin of replication (Ori), an exemplary promoter that may provide a binding site for initiating transcription of a gene of interest (e.g., an exemplary truncated HAS gene set forth in SEQ ID NO: 3), a ribosomal binding site (RBS), one or more exemplary regulatory elements, a Poly-A (polyadenylation) tail (adapted to protect an mRNA from degradation by nucleases and for terminating translation and/or transcription procedures), an exemplary transcription termination site, an exemplary reporter gene (adapted to produce a reporter protein that may be identified and/or quantified with an exemplary assay), and an exemplary multiple cloning site (MCS)/polylinker having one or more exemplary restriction sites. Cloning site, multiple cloning site (MCS), or polylinker may refer to a segment on a vector that may be designed with the purpose of inserting one or more nucleic acid sequences into a vector of interest—for example, to insert a nucleic acid sequence having an ORF encoding a certain polypeptide (e.g., the polynucleotide (SEQ ID NO: 3) encoding an exemplary truncated HAS set forth in SEQ ID NO: 2). In one or more exemplary embodiments, an exemplary promoter may include—but is not limited to—trp, lac, tac, GAP (glucose aldehyde 3-phosphate), λPL, AOX1, GAL10, GAL1, nmt1, nmt81, and nmt42 promoters. An exemplary antibiotic-resistance marker may provide antibiotic-resistance to an exemplary bacteria harboring an exemplary antibiotic-resistance marker and may facilitate detection of an exemplary transformed bacteria on an exemplary selective media (i.e., an exemplary antibiotic-supplemented growth media).

In one or more exemplary embodiments, an exemplary reporter gene may be employed to detect intracellular localization of an exemplary expressed protein (e.g., an exemplary truncated HAS set forth in SEQ ID NO: 2), and also to measure efficiency of gene expression. An exemplary reporter gene may include—but is not limited to—lac Z gene, Luciferase encoding gene, CAT (chloramphenicol acetyltransferase) gene, etc. In one or more exemplary embodiments, one or more exemplary sequence tags may be used for purification, detection, and/or localization of an exemplary protein of interest (e.g., an exemplary truncated HAS set forth in SEQ ID NO: 2).

With further reference to FIG. 2, step 206 may include transferring exemplary expression vector into exemplary host cell to express exemplary truncated HAS (SEQ ID NO: 2). Expressing exemplary truncated HAS may refer to synthesis of exemplary truncated HAS inside an exemplary host cell such as a yeast, bacteria, animal, or a plant cell. For example, in an exemplary embodiment, an exemplary host cell/organism may be an exemplary E. coli strain. An exemplary E. coli strain may include, but is not limited to, ER2566, GI698, B834 (DE3), BL21 (DE3), M15, BLR (DE3), and the like that are known in the art and may be available on the market.

In one or more exemplary embodiments, transferring exemplary expression vector into exemplary host cell/organism may be accomplished using any method by which nucleic acids may be transferred into an exemplary host cell/organism. Exemplary methods may include, but are not limited to, protoplast fusion, electroporation, calcium chloride (CaCl₂) precipitation, calcium phosphate (CaPO₄) precipitation, PEG-mediated transformation, lipofectamine-mediated transformation, agrobacterium-mediated transformation, dextran sulfate-mediated transformation, and desiccation/inhibition-mediated transformation, and agitation with silicon carbide fiber, etc.

Step 208 may include extracting and purifying exemplary truncated HAS (SEQ ID NO: 2) from exemplary host cell/organism using an exemplary extraction and purification method. In an exemplary embodiment, extraction of exemplary truncated HAS (SEQ ID NO: 2) may be accomplished by disrupting exemplary host cell/organism using an exemplary technique including, but not limited to, ultrasonic treatment, homogenizer disrupting, high pressure extrusion, grinding, lysozyme treatment, etc. In one or more exemplary embodiments, exemplary purification method set forth in step 208 may include, but is not limited to, ultracentrifugation, precipitation and differential solubilization, chromatography, and gradient centrifugation. In one or more exemplary embodiments, step 208 may further include removing reductant from exemplary truncated HAS (SEQ ID NO: 2) using an exemplary method including, but not limited to, ultrafiltration, dialysis, and chromatography.

It is to be understood that, in one or more exemplary embodiments, exemplary amino acid sequences may be prepared by in vitro transcription/translation or recombinantly. Exemplary amino acid sequences may also be obtained synthetically, e.g., using a commercially available peptide synthesizer. Methods of synthetic peptide synthesis may include, but are not limited to, solid-phase peptide synthesis, liquid-phase peptide synthesis, etc.

Exemplary nucleic acid molecules/sequences described above may be non-naturally occurring. One or more exemplary embodiments may provide exemplary nucleic acid molecules in synthetic, recombinant, isolated, and/or purified form. Synthetic may refer to exemplary polynucleotides prepared, produced, and/or manufactured by the hand of man. Synthesis of exemplary polynucleotides may be enzymatic or chemical.

### EXAMPLES

Hereinafter, exemplary embodiments will be described in further detail with reference to examples. It will be obvious to a person having ordinary skill in the art that these examples may be for illustrative purposes only and are not to be interpreted to limit the scope of the present disclosure.

### Example 1: Obtaining an Exemplary Polynucleotide Encoding an Exemplary Truncated Hyaluronan Synthase (HAS)

In this example, an exemplary polynucleotide encoding an exemplary truncated HAS (SEQ ID NO: 2) was obtained using an exemplary full-length/native HAS gene—set forth in SEQ ID NO: 4—as template. In an exemplary embodiment, exemplary polynucleotide encoding exemplary truncated HAS (SEQ ID NO: 2) may have an exemplary nucleic acid sequence set forth in SEQ ID NO: 3.

In this example, Streptococcus dysgalactiae subspecies equisimilis—belonging to group G of Streptococcus (GGS)—may be used to express an exemplary truncated HAS (SEQ ID NO: 2). In one or more exemplary embodiments, an exemplary native gene and protein expressed by Streptococcus dysgalactiae subspecies equisimilis may be referred to as full-length native HAS, native full-length HAS, native HAS, full-length HAS, GGS-S88 HAS, GGS-HAS, and any equivalents thereof.

To obtain exemplary polynucleotide (SEQ ID NO: 3) encoding exemplary truncated HAS (SEQ ID NO: 2), GGS equisimilis isolate S88 (GGS-S88) was cultured on a brain-heart infusion medium at 37 ° C. Then, an exemplary genomic DNA of GGS-S88 isolate was extracted using a DNA extraction kit. Since no verified sequence has been published for exemplary GGS-S88 genome, exemplary nucleic acid sequence of exemplary HAS of S. zooepidemicus (GenBank: AF023876.1) was used as a reference to design oligonucleotide primers capable of amplifying exemplary full-length HAS gene of GGS-S88 (SEQ ID NO: 4). Exemplary primers used for amplifying exemplary full-length HAS gene are set forth in SEQ ID NOs: 5 and 6 (exemplary forward and reverse primers, respectively), and exemplary primers used for amplifying exemplary truncated HAS are set forth in SEQ ID NOs: 7 and 8 (exemplary forward and reverse primers, respectively). Table 1 below sets forth exemplary primers used for amplifying exemplary full-length HAS (SEQ ID NO: 4) and an exemplary truncated form of exemplary full-length HAS (SEQ ID NO: 3), consistent with one or more exemplary embodiments of the present disclosure. Capitalized letters in the primer sequences set forth in Table 1 may refer to exemplary restriction sites which may be identified by Ndel and Xhol restriction enzymes.

TABLE 1
Exemplary primers used for amplifying exemplary
full-length HAS (SEQ ID NO: 4) and exemplary
truncated form of exemplary full-length HAS
(SEQ ID NO: 3) through polymerase chain reaction
(PCR), consistent with one or more exemplary
embodiments of the present disclosure.

| | | Primer's | |
|---|---|---|---|
| Title of | Forward/ | Sequence | |
| PCR | Reverse | ID | |
| Product | Primer | Number | Sequence (5′ to 3′) |
| Exemplary | Forward | SEQ ID | ggaattcCATATGagaaca |
| full-length | | NO: 5 | ttaaaaaacctcataactg |
| HAS | | | |
| Exemplary | Reverse | SEQ ID | cccgCTCGAGtaataattt |
| full-length | | NO: 6 | tttacgtgttcc |
| HAS | | | |
| Exemplary | Forward | SEQ ID | tacCATATGaagccattta |
| truncated | | NO: 7 | agggaagg |
| HAS | | | |
| Exemplary | Reverse | SEQID | ataCTCGAGaggattgttc |
| truncated | | NO: 8 | atgattttcttaacag |
| HAS | | | |

Amplification of exemplary full-length HAS and exemplary truncated HAS was carried out under the following exemplary program: denaturation at 95° C. for 5 min followed by 30 cycles at 95° C. for 45 sec, annealing at 52° C. for 45 sec, extension at 72° C. for 3 min, and a 10 min final extension at 72° C. Exemplary full-length HAS gene of S. zooepidemicus was used as positive control.

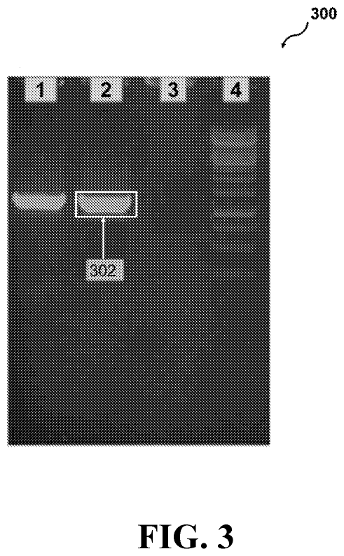

Following exemplary amplification, the obtained PCR (Polymerase Chain Reaction) products were analyzed using 0.7% agarose gel electrophoresis. FIG. 3 shows agarose gel electrophoresis 300 of an exemplary PCR conducted for amplifying exemplary full-length HAS gene of GGS-S88 (SEQ ID NO: 4), consistent with one or more exemplary embodiments of the present disclosure. As shown in FIG. 3, Lane 1 was loaded with the PCR product of an exemplary positive control, i.e., an exemplary sample containing S. zooepidemicus genome. Lane 2 was loaded with the PCR product of an exemplary sample containing exemplary GGS-S88 genome; exemplary band containing an exemplary amplified GGS-S88 HAS gene (1271 bp, SEQ ID NO: 4) is numbered as 302 in Lane 2 of FIG. 3. Lanes 3 and 4 were loaded with an exemplary 1 Kb DNA ladder and a negative control sample, respectively.

An exemplary PCR-product purification kit was used to purify exemplary PCR product of exemplary GGS-S88 HAS gene (SEQ ID NO: 4). Exemplary GGS-S88 HAS gene was suitable to be cloned into an exemplary expression vector like pET21a(+) plasmid after performing an exemplary purification step.

To perform an exemplary cloning step, exemplary PCR product of exemplary GGS-S88 HAS gene and pET21a(+) were both double digested using NdeI and XhoI restriction enzymes. Subsequently, exemplary double digested genes and vectors were purified using an exemplary DNA purification kit. Ligation was performed at a 1:3 vector/insert ratio to completely insert exemplary GGS-S88 HAS gene into an exemplary pET21a(+). Then, exemplary recombinant pET21a(+) plasmid—harboring exemplary GGS-S88 HAS gene—was transformed into an exemplary Escherichia coli Top10 (E. coli Top10) using an exemplary heat-shock technique. An exemplary pET21a(+) lacking exemplary GGS-S88 HAS gene was used as an exemplary negative control.

Colony PCR, double digestion, and sequencing methods were all used to confirm cloning of exemplary GGS-S88 HAS gene into exemplary pET21a(+). An exemplary Colony PCR was conducted using an exemplary T7 promoter (as an exemplary forward primer) and an exemplary reverse primer set forth in SEQ ID NO: 6 under the following exemplary program: denaturation at 96° C. for 5 min followed by 30 cycles at 95° C. for 45 sec, annealing at 54° C. for 45 sec, and extension at 72° C. for 90 sec, and a 10 min-final extension at 72° C. PCR products were then analyzed using 1% agarose gel electrophoresis.

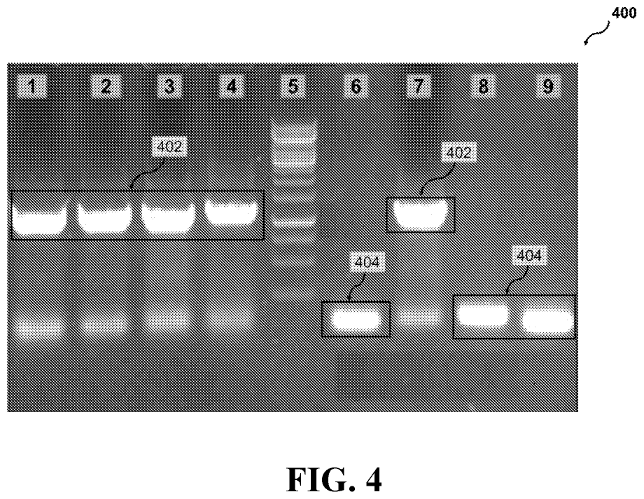

FIG. 4 illustrates agarose gel electrophoresis 400 of an exemplary colony PCR conducted on exemplary E. coli Top10 colonies that were transformed with exemplary recombinant pET21a(+) vector (harboring exemplary GGS-S88 HAS gene), consistent with one or more exemplary embodiments of the present disclosure. As shown in FIG. 4, Lanes 1-4 and Lane 7 represent positive colonies containing exemplary full-length HAS gene (GenBank: AF023876.1) of S. zooepidemicus, Lane 5 was loaded with an exemplary 1 Kb DNA ladder, and Lanes 6, 8 and 9 represent exemplary GGS-S88 HAS gene (1271 bp, SEQ ID NO: 4). In FIG. 4, exemplary electrophoresis bands containing exemplary full-length HAS gene of S. zooepidemicus is numbered as 402, and exemplary bands containing exemplary GGS-S88 HAS gene (1271 bp, SEQ ID NO: 4) are numbered as 404.

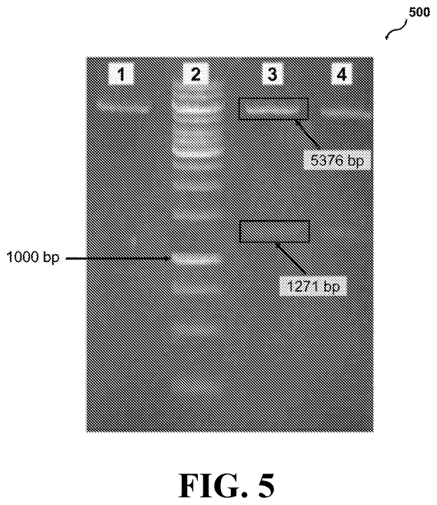

FIG. 5 shows restriction map analysis 500 of exemplary recombinant plasmids (i.e., exemplary pET21a(+) harboring exemplary GGS-S88 HAS gene) using NdeI and XhoI restriction enzymes, to verify insertion of exemplary GGS-S88 HAS gene into exemplary pET21a(+) vector, consistent with one or more exemplary embodiments of the present disclosure. As shown in FIG. 5, Lane 1 represents an exemplary negative control (i.e., an exemplary pET21a(+) lacking exemplary GGS-S88 HAS gene insert), Lane 2 was loaded with an exemplary 1 Kb DNA ladder, Lane 3 constitutes exemplary recombinant pET21a(+) vector harboring exemplary GGS-S88 HAS gene (SEQ ID NO: 4), and Lane 4 represents an exemplary positive control (i.e., exemplary pET21a(+) vector harboring exemplary HAS gene of S. zooepidemicus).

After sequencing the inserted exemplary GGS-S88 HAS gene in exemplary pET21a(+), an exemplary multiple alignment was conducted to align exemplary nucleic acid sequence of exemplary GGS-S88 HAS gene (e.g., SEQ ID NO: 4) with similar sequences including exemplary full-length/native HAS gene of Streptococcus equisimilis group C (GCS, GenBank: AF023876.1) and exemplary full-length/native HAS gene of Streptococcus equisimilis group A (GAS, GenBank: L21187.1). Sequence alignment of exemplary GGS-S88 HAS gene with exemplary GCS HAS gene (GenBank: AF023876.1) and exemplary GAS HAS gene (GenBank: L21187.1) showed 98.40% and 62.18% identity, respectively. According to the accomplished multiple alignment, exemplary full-length/native HAS gene of GGS-S88 (SEQ ID NO: 4) may have a size of about 1371 bp and may encode an exemplary GGS-S88 HAS with about 417 amino acid residues (SEQ ID NO: 1).

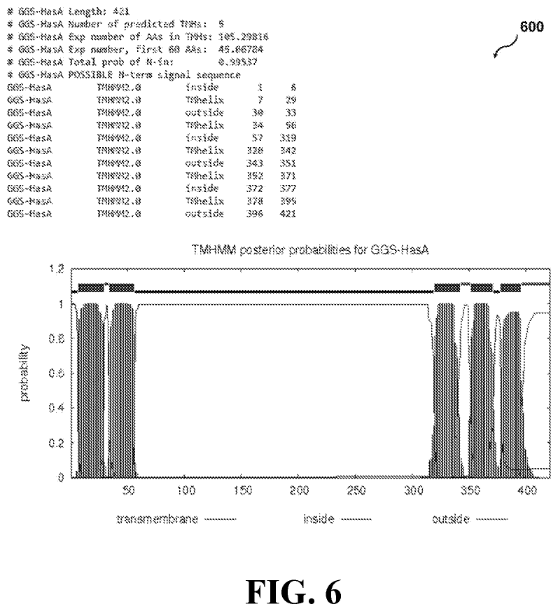

An exemplary membrane protein topology prediction method, i.e., an exemplary TMHMM method, was employed to predict exemplary transmembrane helices of exemplary GGS-S88 HAS. FIG. 6 shows topology prediction result 600 for exemplary native full-length HAS (i.e., exemplary GGS-S88 HAS) obtained using the TMHMM server, consistent with one or more exemplary embodiments of the present disclosure. Based on topology prediction result 600, exemplary GGS-S88 HAS (referred to as exemplary native full-length HAS in one or more exemplary embodiments) may have five exemplary membrane-associated domains (MDs), three exemplary extracellular regions (localized outside an exemplary cell), and three exemplary intracellular regions (localized inside an exemplary cell). As stated in the “DETAILED DESCRIPTION” section, FIG. 1 shows exemplary schematic structure 100 of GGS-588, consistent with one or more exemplary embodiments of the present disclosure. In an exemplary embodiment, i) residues 1-6, 57-319, and 372-377 of SEQ ID NO: 1 may, respectively, compose exemplary first intracellular region 101, exemplary second intracellular region 105, and exemplary third intracellular region 109 of exemplary native full-length HAS (SEQ ID NO: 1); ii) residues 7-29, 34-56, 320-342, 352-371, and 378-395 may, respectively, compose exemplary MD1 (102), exemplary MD2 (104), exemplary MD3 (106), exemplary MD4 (108), and exemplary MD5 (110) of exemplary native full-length HAS (SEQ ID NO: 1); and iii) residues 30-33, 343-351, and 396-417 may, respectively, compose exemplary first extracellular region 103, exemplary second extracellular region 107, and exemplary third extracellular region 112 of exemplary native full-length HAS (SEQ ID NO: 1). An exemplary truncated HAS (SEQ ID NO: 2) may lack all of the transmembrane/membrane-associated domains (MDs), compared to exemplary native full-length HAS (i.e., exemplary GGS-S88 HAS). This property may solve exemplary complications associated with in vitro production of hyaluronic acid (HA) using exemplary full-length HAS which needs to be localized inside an exemplary plasma membrane in order to be capable of producing an exemplary HA. Table 2 bellow sets forth exemplary characteristics of exemplary truncated HAS (SEQ ID NO: 2) and exemplary domains that exemplary truncated HAS may lack, compared to exemplary native full-length HAS (SEQ ID NO: 1).

TABLE 2
Characteristics of exemplary truncated HAS having exemplary amino acid sequence set forth
in SEQ ID NO: 2 and exemplary domains that exemplary truncated HAS may lack, compared
to exemplary native full-length HAS, consistent with one or more exemplary embodiments of
the present disclosure.

| | | | | Exemplary removed |
|---|---|---|---|---|
| | | | Position on | membrane- |
| | Length | Sequence | exemplary native | associated domains |
| Title | (residues) | ID number | full-length HAS | (MDs) |
| Exemplary full- | 417 | SEQ ID | | |
| length HAS (i.e., | | NO: 1 | | |
| exemplary GGS- | | | | |
| S88 HAS) | | | | |
| Exemplary | 159 | SEQ ID | Residues 57 to | MD1, MD2, MD3, |
| truncated HAS | | NO: 2 | 215 of SEQ ID | MD4, MD5 |
| | | | NO: 1 | |

Based on exemplary sequenced GGS-S88 HAS gene (SEQ ID NO: 4), an exemplary set of specific primers were designed for amplifying exemplary truncated HAS gene (SEQ ID NO: 3) using Gene Runner© V6.1 software (exemplary forward and reverse primers for amplification of exemplary truncated HAS gene are set forth in SEQ ID NOs: 7 and 8, respectively). Exemplary truncated HAS gene (SEQ ID NO: 3) may encode exemplary amino acid sequence set forth in SEQ ID NO: 2.

PCR, for amplification of exemplary truncated HAS gene (SEQ ID NO: 3), was conducted on an exemplary “GGS-588 HAS”-harboring pET21a(+) using Pfu DNA polymerase under the following exemplary program: denaturation at 95° C. for 5 min followed by 30 cycles at 95° C. for 30 sec, annealing at 55° C. for 30 sec, extension at 72° C. for 2 min, and a 10 min final extension at 72° C.

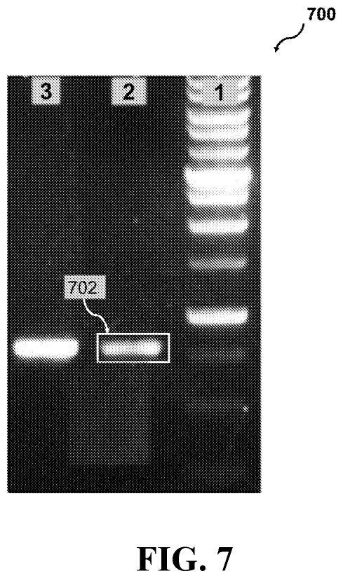

FIG. 7 shows agarose gel electrophoresis 700 of an exemplary PCR for amplification of exemplary truncated HAS gene (SEQ ID NO: 3), consistent with one or more exemplary embodiments of the present disclosure. Lane 1 was loaded with an exemplary 1 Kb DNA ladder, Lane 2 was loaded with the PCR product of an exemplary sample containing exemplary “GGS-588 HAS”-harboring pET21a(+) (amplified using exemplary forward and reverse primers set forth in SEQ ID NOs: 7 and 8, respectively), and Lane 3 was loaded with the PCR product of an exemplary positive control sample. Label 702 in FIG. 7 shows an exemplary amplified truncated HAS gene (807 bp).

### Example 2: Expression, Identification, and Purification of Exemplary Full-Length HAS and an Exemplary Truncated Form of Exemplary Full-Length HAS

In this example, an exemplary truncated HAS (SEQ ID NO: 2) was recombinantly produced, verified, and purified. To this aim, cloning of exemplary truncated HAS gene (SEQ ID NO: 3) was performed using the same procedure as previously described for exemplary full-length HAS gene in “Example 1”.

Exemplary recombinant plasmids (pET21a(+)) harboring exemplary truncated HAS gene (SEQ ID NO: 3) were isolated and transformed into an exemplary E. coli BL21 (DE3) strain. An exemplary truncated HAS (SEQ ID NO: 2) was expressed in an exemplary 2xYT broth medium containing ampicillin (100 μg/ml), 1 mM IPTG (Isopropyl β- d-1-thiogalactopyranoside) at A600 of 0.7, at 37° C. for 24 h. A same protein-expression procedure was accomplished for an exemplary negative control (i.e., an exemplary bacterial colony containing an exemplary pET21a(+) that may lack exemplary GGS-S88 HAS gene). 1 ml of exemplary bacterial cultures were collected before and after IPTG induction for measuring bacterial growth (by measuring optical density (OD) at 600 nm), and for analyzing expressed proteins by sodium dodecyl sulfate-polyacrylamide gel electrophoresis (SDS-PAGE, 12%) and western blotting. Exemplary cloning steps were confirmed by performing an exemplary colony PCR and exemplary double digestion methods.

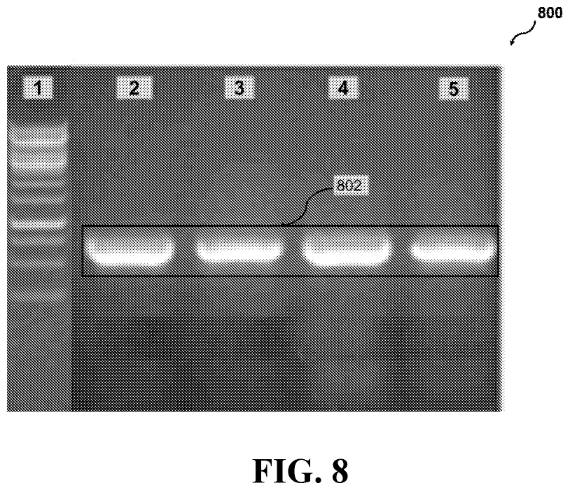

FIG. 8 illustrates agarose gel electrophoresis 800 of an exemplary colony PCR conducted on exemplary E. coli BL21 (DE3) colonies that were transformed with exemplary pET21a(+) vectors harboring exemplary truncated HAS gene (SEQ ID NO: 3), consistent with one or more exemplary embodiments of the present disclosure. Lane 1 was loaded with an exemplary DNA ladder (1 Kb), and Lanes 2-5 comprise exemplary truncated HAS gene (807 bp, labeled as 802).

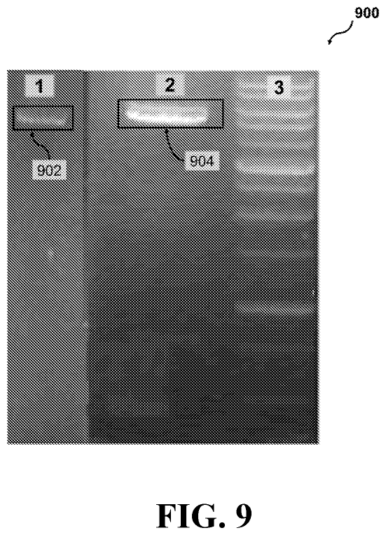

FIG. 9 shows restriction map analysis 900 of exemplary recombinant plasmids (i.e., exemplary pET21a(+) vectors harboring exemplary truncated HAS gene), using NdeI and XhoI restriction enzymes, to verify insertion of exemplary truncated HAS gene (SEQ ID NO: 3) into exemplary pET21a(+) vector, consistent with one or more exemplary embodiments of the present disclosure. Lane 1 represents an exemplary positive control (i.e., exemplary pET21a(+) vector harboring exemplary S. zooepidemicus' HAS gene) labeled as 902, Lane 2 constitutes exemplary pET21a(+) harboring exemplary truncated HAS gene (SEQ ID NO: 3) labeled as 904, and Lane 3 was loaded with an exemplary 1 Kb DNA ladder.

After conducting an exemplary protein-expression step, exemplary recombinant cells were pelleted by centrifugation at 4° C. for 30 min at 3000 g. Exemplary pellets were washed twice with Phosphate-Buffered Saline (PBS) containing 1.3 M glycerol at 4° C., followed by getting frozen at −80° C. Purification of exemplary plasma membranes containing exemplary truncated HAS (SEQ ID NO: 2) was accomplished by employing an exemplary protoplast method. Briefly, after thawing exemplary frozen pellets, exemplary pellets were resuspended in an exemplary solution—containing 30 mM Tris (pH=8.2), 10 mM MgCl₂, 20% sucrose, 1 mM dithiothreitol, 46 μg/ml phenylmethylsulfonyl fluoride (PMSF), and lysozyme (20 mg/ml)—and were subsequently incubated on ice for 40 min. After sonication, exemplary cell lysates were diluted 2-folds in PBS buffer containing 1.3 M glycerol, 60 mM MgCl₂, 1 mM dithiothreitol, 46 μg/ml PMSF, 1 μg/ml DNase, and 1 μg/ml Rnase, and were mixed on ice for 20 min. Following centrifugation at 10000 g for 30 min at 4° C., cell debris was removed and exemplary plasma membranes were deposited by ultracentrifugation at 100000 g for 1 h. Exemplary plasma membranes were then resuspended with PBS (containing 1.3 M glycerol and 46 μg/ml PMSF), sonicated, and re-centrifuged at 100000 g for 1 h. Finally, Exemplary plasma membranes were stored at −80° C. for further protein purification.

Purification of exemplary full-length HAS (SEQ ID NO: 1) and exemplary truncated HAS (SEQ ID NO: 2) was carried out using an exemplary method. Briefly, exemplary plasma membranes were thawed, suspended, and mixed in 10 ml of an exemplary extraction buffer (50 mM sodium and potassium phosphate, 10 mM MgCl₂, 150 mM NaCl, 46 μg/ml PMSF, and 1 mM β-mercaptoethanol, adjusted to pH=7) for 2 h at 4 ° C. on a rotating shaker, followed by centrifugation at 100000 g for 1 h at 4° C.

After resuspension in 30 mM imidazole, exemplary mixtures that were prepared as above were incubated with 0.3 ml Ni²⁺-NTA (Nitrilotriacetic acid) resin-equilibrated with an exemplary extraction buffer lacking MgCl₂—at 4° C. with constant mixing for 2 h. The resin was washed with exemplary extraction buffer and exemplary enzymes (i.e., exemplary full-length and exemplary truncated HAS) were eluted using an exemplary elution buffer (comprising 200 mM imidazole, 25 mM sodium and potassium phosphate, 50 mM NaCl, 46 μg/mL PMSF, 2.7 M glycerol, and 1 mM dithiothreitol, adjusted to pH=7).

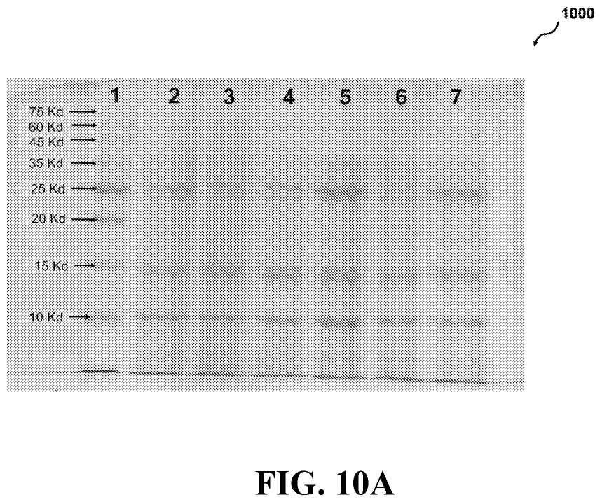

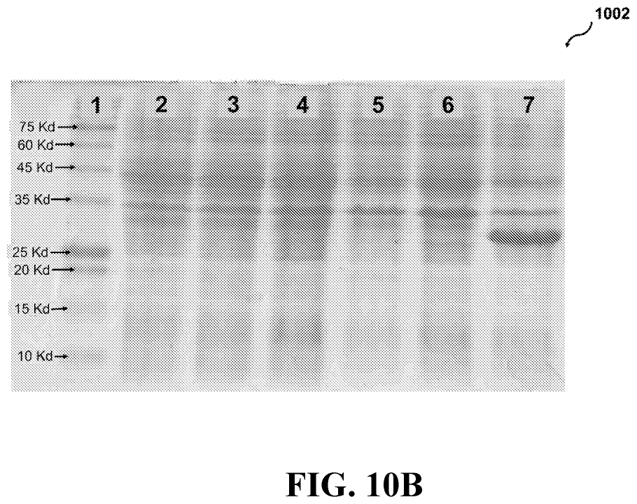

Exemplary eluted enzymes were then analyzed by 12% SDS-PAGE. FIG. 10A shows SDS-PAGE analysis 1000 of exemplary purified full-length HAS and exemplary purified truncated HAS (SEQ ID NOs: 2 and 1, respectively) before IPTG induction, consistent with one or more exemplary embodiments of the present disclosure. FIG. 10B shows SDS-PAGE analysis 1002 of exemplary purified full-length HAS and exemplary purified truncated HAS (SEQ ID NOs: 2 and 1, respectively) after IPTG induction, consistent with one or more exemplary embodiments of the present disclosure. Lane 1 of FIG. 10A and FIG. 10B shows an exemplary 10-170 kDa pre-stain protein marker. Referring to FIG. 10B, Lane 6 comprises exemplary truncated HAS protein band (30 kDa) and Lane 7 comprises the protein band of exemplary full-length HAS protein (42 kDa).

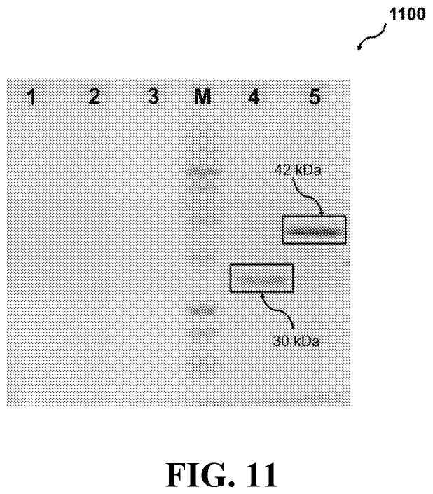

Expression of exemplary eluted enzymes (i.e., exemplary purified full-length and exemplary purified truncated HAS (SEQ ID NOs: 2 and 1, respectively)) was confirmed by western blotting using an exemplary anti-polyhistidine HRP (Horse Raddish Peroxidase). Briefly, after separating exemplary protein mixture by SDS-PAGE, exemplary separated protein bands were transferred from exemplary SDS-PAGE to an exemplary PVDF (Polyvinylidene difluoride) membrane using an exemplary semi-dry electrophoresis. Blockade of exemplary PVDF membrane was accomplished using 5% BSA diluted in TBST buffer (solution of NaCl, KCl, Tris base, and Tween 20 in distilled H₂O). After washing exemplary PVDF membrane with TBST, it was incubated with monoclonal anti-polyhistidine HRP for 1-2 h. The blotting result was visualized by adding DAB (3,3′-diaminobenzidine, Sigma, Germany) substrate to the surface of exemplary PVDF membrane at dark. FIG. 11 shows western blot analysis 1100 of exemplary full-length HAS and exemplary truncated HAS (SEQ ID NOs: 2 and 1, respectively) after expression in exemplary transformed E. coli BL21 (DE3), consistent with one or more embodiments of the present disclosure. As mentioned above, a first group of exemplary transformed E. coli BL21 (DE3) cells may contain exemplary recombinant vectors (i.e., exemplary pET21a(+)) harboring exemplary full-length HAS polynucleotide set forth in SEQ ID NO: 4 and a second group of exemplary transformed E. coli BL21 (DE3) cells may contain exemplary recombinant vectors (i.e., exemplary pET21a(+)) harboring exemplary truncated HAS polynucleotide set forth in SEQ ID NO: 3. As shown in FIG. 11, Lane M shows an exemplary protein molecular weight marker (10-180 kDa); Lane 1-2 comprises exemplary bacterial pellet carrying exemplary “full-length HAS”-pET21a(+) recombinant vector before IPTG induction; Lane 3 comprises exemplary bacterial pellet carrying exemplary “truncated HAS”-pET21a(+) recombinant vector before IPTG induction; Lane 4 comprises exemplary bacterial pellet carrying exemplary “truncated HAS”-pET21a(+) recombinant vector after IPTG induction; and Lane 5 comprises exemplary bacterial pellet carrying exemplary “full-length HAS”-pET21a(+) recombinant vector after IPTG induction. The western blot analysis revealed an exemplary 30 kDa protein band (i.e., exemplary truncated HAS protein band) in Lane 4 and an exemplary 42 kDa protein band (i.e., exemplary full-length HAS protein band) in Lane 5.

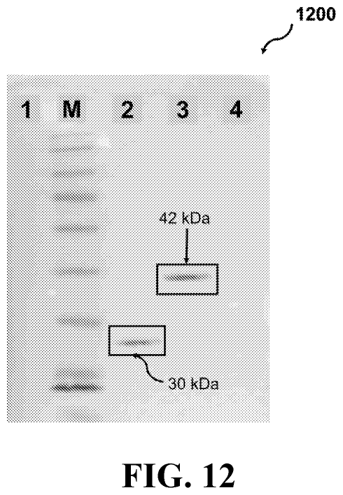

FIG. 12 shows western blot analysis 1200 of exemplary full-length HAS and exemplary truncated HAS (SEQ ID NOs: 2 and 1, respectively) after being eluted from an exemplary Ni²⁺-NTA column, consistent with one or more embodiments of the present disclosure. As shown in FIG. 12, Lane M is an exemplary protein molecular weight marker (10-180 kDa); Lane 1 represents exemplary bacterial pellet containing exemplary “truncated HAS”-pET21a(+) recombinant vector before IPTG induction (as negative control); Lane 2 was loaded with exemplary eluted truncated HAS (containing a 30 kDa protein band); Lane 3 was loaded with exemplary eluted full-length HAS (containing a 42 kDa protein band); and Lane 4 represents exemplary bacterial pellet containing exemplary “full-length HAS”-pET21a(+) recombinant vector before IPTG induction (as an exemplary negative control).

### Example 3: Evaluation of Enzymatic Activity of Exemplary Truncated HAS

Prior to measuring enzyme activity of exemplary full-length HAS and exemplary truncated HAS, absorbance of exemplary purified HAS enzymes (i.e., exemplary full-length HAS and exemplary truncated HAS) was measured at 280 nm. In this example, enzyme activity was evaluated—in triplicate—by mixing 100 μl of a polymerization reaction buffer (containing 1 mM UDP-GlcUA and 1 mM UDP-GlcNAc, 25 mM sodium and potassium phosphate, 1 mM dithiothreitol, 50 mM NaCl, 20 mM MgCl₂, 2 M glycerol, and 1 mM EDTA, adjusted to pH=7) with 100 μl of exemplary purified enzymes at 37° C. for 1 h with constant shaking.

In this example, an exemplary sensitive 96-well assay of uronic acid was used to determine complex uronic acid-bearing polyanions, such as HA. Meanwhile, an exemplary reaction buffer solution lacking both of exemplary enzymes and substrate, and an exemplary reaction buffer solution lacking exemplary enzymes were used as exemplary negative controls.

To perform 96-well assay of uronic acid, a serial dilution of exemplary glucuronic acid standard solutions was prepared (0 to 100 μg/ml). Then, 50 μl of exemplary standard solutions and exemplary enzymatic reactions were separately dropped into the wells of an exemplary 96-well plate. Then, 200 μl of an exemplary 25 mM sodium tetraborate solution in sulfuric acid was added to each well and heated at 80° C. for 20 min in an oven. After cooling exemplary plates by placing them at 25° C. for 15 minutes, 50 μl of an exemplary 0.125% carbazole in absolute ethanol was added to each well. Exemplary plates were reheated again and, after being cooled at 25° C., the absorbance of each well was measured at the wavelength of 550 nm—for both of exemplary standard solutions and exemplary enzymatic reactions using an ELISA (enzyme-linked immunosorbent assay) reader. The amount of HA production in exemplary enzymatic reactions was calculated using an exemplary standard curve plotted based on exemplary standard solutions.

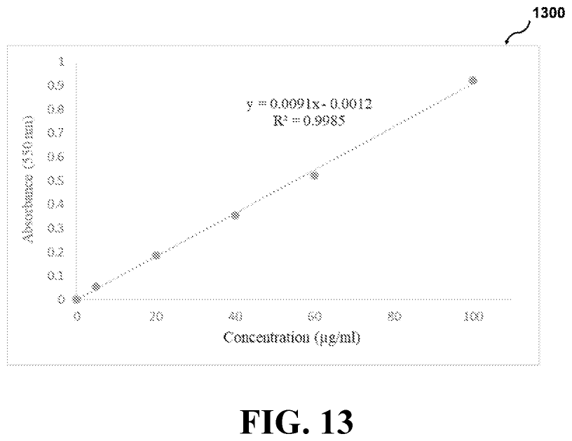

FIG. 13 shows an exemplary standard curve 1300 obtained based on measuring the absorbance of exemplary glucuronic acid standard solutions in an exemplary carbazole assay (at 550 nm), consistent with one or more exemplary embodiments of the present disclosure. Table 3 below shows the obtained absorbance for a plurality of exemplary standard solutions containing different concentrations of an exemplary glucuronic acid (each concentration being in triplicate).

TABLE 3
Absorbance of exemplary glucuronic acid standard solutions, containing 0 to 100 μg/ml of an
exemplary glucuronic acid, at 550 nm (in triplicate).

| Glucuronic acid | 100 | 60 | 40 | 20 | 5 | |
|---|---|---|---|---|---|---|
| concentration | μg/ml | μg/ml | μg/ml | μg/ml | μg/ml | Blank |
| Absorbance (1) | 0.548 | 0.439 | 0.379 | 0.226 | 0.134 | 0.101 |
| Absorbance (2) | 0.555 | 0.369 | 0.295 | 0.272 | 0.129 | 0.128 |
| Absorbance (3) | 0.499 | 0.342 | 0.293 | 0.246 | 0.123 | 0.104 |

Table 4 bellow sets forth an exemplary HA-synthesis activity of exemplary truncated HAS (SEQ ID NO: 2) and exemplary full-length HAS (SEQ ID NO: 1), consistent with one or more exemplary embodiments of the present disclosure. Exemplary truncated HAS (SEQ ID NO: 2) demonstrated an acceptable HA-synthesis activity, whereas exemplary full-length HAS (SEQ ID NO: 1) had a higher enzyme activity as expected.

TABLE 4
An exemplary HA-synthesis activity of exemplary full-length HAS and exemplary truncated
HAS (SEQ ID NOs: 2 and 1, respectively), consistent with one or more exemplary
embodiments of the present disclosure.

| | | | | | % Productivity of |
|---|---|---|---|---|---|
| | | | | Activity | exemplary truncated |
| | | Hyaluronic | | (μg HA/ | HAS/% Productivity |
| | Sequence ID | Acid | Enzyme | mg | of exemplary full- |
| Title | Number | (μg/mL) | (mg/mL) | Enzyme) | length HAS |
| Exemplary | SEQ ID NO: | 417.05 | 0.551 | 756.9 | 100 |
| full-length | 1 | | | | |
| HAS | | | | | |
| Exemplary | SEQ ID NO: | 68.96 ± 0.951 | 0.532 | 128.8 ± 0.458 | 17 |
| truncated | 2 | | | | |
| HAS | | | | | |

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. An element proceeded by “a” or “an” does not, without further constraints, preclude the existence of additional identical elements in the process, method, article, or apparatus that comprises the element.

Unless otherwise stated, all measurements, values, ratings, positions, magnitudes, sizes, and other specifications that are set forth in this specification, are approximate, not exact. They are intended to have a reasonable range that is consistent with the functions to which they relate and with what is customary in the art to which they pertain.

It will be understood that the terms and expressions used herein have the ordinary meaning as is accorded to such terms and expressions with respect to their corresponding respective areas of inquiry and study, except where specific meanings have otherwise been set forth herein. Relational terms such as “first” and “second” and the like may be used solely to distinguish one entity or action from another without necessarily requiring or implying any actual such relationship or order between such entities or actions.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it may be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A truncated hyaluronan synthase (HAS) comprising an amino acid sequence having at least 95% identity with SEQ ID NO: 2.

2. The truncated HAS of claim 1, wherein the truncated HAS is encoded by the nucleic acid sequence set forth in SEQ ID NO: 3.


SEQUENCE LISTING (8 sequences)

SEQ ID NO: 1 - protein, 417 aa, Streptococcus dysgalactiae subspecies equisimilis
  TRANSMEM (7)..(29): MD1 (i.e., membrane-associated domain 1)
  TRANSMEM (34)..(56): MD2 (i.e., membrane-associated domain 2)
  TRANSMEM (320)..(342): MD3 (i.e., membrane-associated domain 3)
  TRANSMEM (352)..(371): MD4 (i.e., membrane-associated domain 4)
  TRANSMEM (378)..(395): MD5 (i.e., membrane-associated domain 5)
  ref <308>: DATABASE ACCESSION NUMBER: GenBank/QSG30229.1
  ref <309>: DATABASE ENTRY DATE: 2021-03-06
  ref <313>: RELEVANT RESIDUES IN SEQ ID NO: (5)..(421)
LKNLITVVAFSIFWVLLIYVNVYLFGAKGSLSIYGFLLIAYLLVKMSLSFFYKPFKGRAGQYKVAAIIPSYNEDAESLLETLKSVQQQTYPLAEIYVVDDGSADETGIKRIEDYVRETGDLSSNVIVHRSEKNQGKRHAQAWAFERSDADVFLTVDSDTYIYPDALEELLKTFNDPTVFAATGHLNVRNRQTNLLTRLTDIRYDNAFGVERAAQSVTGNILVCSGPLSVYRREVVVPNIDRYINQTFLGIPVSIGDDRCLTNYATDLGKTVYQSTAKCITDVPDKMSTYLKQQNRWNKSFFRESIISVKKIMNNPFVALWTILEVSMFMMLVYSVVDFFVGNVREFDWLRVLAFLVIIFIVALCRNIHYMLKHPLSFLLSPFYGVLHLFVLQPLKLYSLFTIRNADWGTRKKLLLER

SEQ ID NO: 2 - protein, 159 aa, Unknown
  Truncated hyaluronan synthase (HAS), expressed in Escherichia coli BL21
PFKGRAGQYKVAAIIPSYNEDAESLLETLKSVQQQTYPLAEIYVVDDGSADETGIKRIEDYVRETGDLSSNVIVHRSEKNQGKRHAQAWAFERSDADVFLTVDSDTYIYPDALEELLKTFNDPTVFAATGHLNVRNRQTNLLTRLTDIRYDNAFGVERA

SEQ ID NO: 3 - DNA, 482 bp, Unknown
  Nucleic acid sequence of the truncated hyaluronan synthase (HAS) gene that may encode the truncated HAS set forth in SEQ ID NO: 1
aagccatttaagggaagggctgggcaatataaggttgcagccattattccctcttataacgaagatgctgagtcattgctagagaccttaaaaagtgttcagcagcaaacctatcccctagcagaaatttatgttgttgacgatggaagtgctgatgagacaggtattaagcgcattgaagactatgtgcgtgaaactggtgacctatcaagcaatgtcattgttcaccggtcagagaaaaatcaaggaaagcgtcatgcacaggcctgggcctttgaaagatcagacgctgatgtctttttgaccgttgactcagatacttatatctaccctgatgctttagaggagttgttaaaaacctttaatgacccaactgtttttgctgcgacgggtcatcttaatgtcagaaatagacaaaccaatctcttaacacgcttgacagatattcgctatgataatgcttttggtgttgaacgagctgc

SEQ ID NO: 4 - DNA, 1261 bp, Streptococcus dysgalactiae subspecies equisimilis
  ref <308>: DATABASE ACCESSION NUMBER: GenBank/MW285140.1
  ref <309>: DATABASE ENTRY DATE: 2021-03-06
  ref <313>: RELEVANT RESIDUES IN SEQ ID NO: (4)..(1264)
atgagaacattaaaaaacctcataactgttgtggcctttagtattttttgggtactgttgatttacgtcaatgtttatctctttggtgctaaaggaagcttgtcaatttatggctttttactgatagcttacctattagtcaaaatgtccttatcctttttttacaagccatttaagggaagggctgggcaatataaggttgcagccattattccctcttataacgaagatgctgagtcattgctagagaccttaaaaagtgttcagcagcaaacctatcccctagcagaaatttatgttgttgacgatggaagtgctgatgagacaggtattaagcgcattgaagactatgtgcgtgaaactggtgacctatcaagcaatgtcattgttcaccggtcagagaaaaatcaaggaaagcgtcatgcacaggcctgggcctttgaaagatcagacgctgatgtctttttgaccgttgactcagatacttatatctaccctgatgctttagaggagttgttaaaaacctttaatgacccaactgtttttgctgcgacgggtcatcttaatgtcagaaatagacaaaccaatctcttaacacgcttgacagatattcgctatgataatgcttttggtgttgaacgagctgcccaatccgttacaggtaatattctcgtttgctcaggcccgcttagcgtttacagacgcgaggtggttgttcctaacatagatagatacatcaaccagaccttcctgggtattcctgtaagtatcggtgatgacaggtgcttgaccaactatgcaactgatttaggaaagactgtttatcaatccactgctaaatgtattacagatgttcctgacaagatgtctacttacttgaagcagcaaaaccgctggaacaagtccttctttagagagtccattatttctgttaagaaaatcatgaacaatccttttgtagccctatggaccatacttgaggtgtctatgtttatgatgcttgtttattctgtggtggatttctttgtaggcaatgtcagagaatttgattggctcagggttttagcctttctggtgattatcttcattgttgctctttgtcgtaatattcactatatgcttaagcacccgctgtccttcttgttatctccgttttatggggtactgcatttgtttgtcctacagcccttgaaattgtattctctttttactattagaaatgctgactggggaacacgtaaaaaattattactcgagcggg

SEQ ID NO: 5 - DNA, 38 bp, Artificial Sequence
  Forward primer for amplifying the full-length hyaluronan synthase (HAS) gene set forth in SEQ ID NO: 4, using the HAS nucleic acid sequence of S. zooepidemicus (GenBank: AF023876.1) as template
ggaattccatatgagaacattaaaaaacctcataactg

SEQ ID NO: 6 - DNA, 31 bp, Artificial Sequence
  Reverse primer for amplifying the full-length hyaluronan synthase (HAS) gene set forth in SEQ ID NO: 4, using the HAS nucleic acid sequence of S. zooepidemicus (GenBank: AF023876.1) as template
cccgctcgagtaataattttttacgtgttcc

SEQ ID NO: 7 - DNA, 27 bp, Artificial Sequence
  Forward primer for amplifying the truncated hyaluronan synthase (HAS) gene set forth in SEQ ID NO: 3, using the full-length HAS gene set forth in SEQ ID NO: 4 as template
taccatatgaagccatttaagggaagg

SEQ ID NO: 8 - DNA, 35 bp, Artificial Sequence
  Reverse primer for amplifying the truncated hyaluronan synthase (HAS) gene set forth in SEQ ID NO: 3, using the full-length HAS gene set forth in SEQ ID NO: 4 as template
atactcgagaggattgttcatgattttcttaacag